(19) United States
(12) Patent Application Publication
(10) Pub. No.: US 2013/0203054 A1
(43) Pub. Date: Aug. 8, 2013

(54) ANTIVIRAL TREATMENT SUSCEPTIBILITY GENE AND USES THEREOF

(71) Applicant: Alain Dessein, Marseille (FR)

(72) Inventor: Alain Dessein, Marseille (FR)

(73) Assignee: UNIVERSITE D'AIX-MARSEILLE, Marseille (FR)

(21) Appl. No.: 13/695,854

(22) PCT Filed: May 4, 2011
(86) PCT No.: PCT/EP2011/057145

§ 371 Date: Jan. 17, 2013

Related U.S. Application Data
(60) Provisional application No. 61/331,060, filed on May 4, 2010.

(51) Int. Cl. C12Q 1/68

(52) U.S. Cl. CPC C12Q 1/6876
USPC 435/6.11

(57) ABSTRACT

The present invention relates to an in vitro method for determining the likelyhood for a patient affected with a viral infection to respond to a treatment with an antiviral agent and/or an interferon, which method comprises determining alteration in CTGF gene locus or in CTGF expression or in CTGF activity in a biological sample of the patient.

### FIELD OF THE INVENTION

The present invention relates generally to the fields of genetics and medicine. The present invention discloses in particular the identification of an antiviral treatment susceptibility gene, which can be used for predicting the response to antiviral treatment of patients suffering from viral infectious disease, especially hepatitis C. The invention more particularly discloses certain alleles of the CTGF (CCN2) gene on chromosome 6 related to response to antiviral treatment of patients who suffer from viral infection and can be used to select responsive patients.

### BACKGROUND OF THE INVENTION

Hepatitis C is a viral disease affecting the liver, caused by the hepatitis C virus (HCV). It is transmitted principally by blood and affects millions of people around the world. There are two phases in hepatitis C infection. The acute phase occurs during the first 6 months after the infection. Most infected people do not develop any symptom. In a second phase chronic hepatitis C, defined as infection with the HCV persisting more than 6 months develops. Once established, chronic hepatitis C can progress and cause fibrosis, especially hepatic fibrosis, and up to 20% of those infected will progress to cirrhosis within 20 years (Schuppan and al., 2003).

A large number of molecules have been tested for treatment of hepatic fibrosis. For example, corticosteroids have been used to suppress hepatic inflammation in autoimmune and alcoholic hepatitis (Czaja et al., 2003). Ursodeoxycholic acid has proven to increase survival in PBC patients by binding bile acids, and thus also decreasing hepatic inflammation (Poupon et al., 1997). Neutralizing inflammatory cytokines with specific receptor antagonists (TNFalpha, IL-1 receptor antagonists) and prostaglandin E have been tested in murine models, but not yet in humans (Bruck et al., 1997).

The current usual treatment combines interferon gamma (IFN-γ) with ribavirin.

However only a fraction of patients responds to the ribavirin-IFN-γ treatment.

Treatment response partly depends of the HCV genotype. Six different genotypes of HCV have been characterized (Nathan and al., 2010). Patient infected with the HCV genotype 1, the most frequent genotype in the Western world (60±90% of those infected), are particularly poor responders (Nathan and al., 2010). Other factors like stage of fibrosis, alcohol assumption and duration of the infection also affect the response to IFN treatment.

There are several host factors like age, sex and ethnicity which affect the susceptibility to the treatment. Response to ribavirin-IFN-γ treatment depends also of patient's genotype.

On the other hand, the ribavirin-IFN combinaison is not devoid of side effects, including fatigue, influenza-like symptoms, hematologic abnormalities, and neuropsychiatric which occur in 10 to 20% of patients (Fried, 2002).

Furthermore the treatment currently costs 250 US$ per week, knowing the treatment generally last 48 weeks (1 year).

Thus, there is a need for a method for selecting patients who have better chances to respond to a treatment in order to optimize treatment, avoid side effects for non-responders and reduce treatment costs.

Pharmacogenetic tests to select patient for their susceptibility to treatment in numerous pathologies have been described, e.g in WO 00/50639 and US 2005/0282179. However there remains a need for a reliable test for determining whether a patient with a viral infection such as HCV infection will respond to an antiviral treatment.

### SUMMARY OF THE INVENTION

A subject of the invention is an in vitro method for determining the likelyhood for a patient affected with a viral infection to respond to a treatment with an antiviral agent and/or an interferon, which method comprises determining alteration in CTGF gene locus or in CCN2 expression or in the activity of CCN2 encoded product (the CTGF protein) in a biological sample of the patient.

Preferably said alteration is a mutation, an insertion or a deletion of one or more bases. More preferably said alteration is one or several single nucleotide polymorphism(s) (SNPs).

In a particular embodiment said SNP is rs9402373 (SEQ ID NO:1) and the presence of a CC genotype of rs9402373 is indicative of a non-responder to said treatment.

In another particular embodiment, said SNP is rs6918698 (SEQ ID NO: 2), and the presence of CC or GG genotype of rs6918698 is indicative of a non-responder to said treatment.

In a particular embodiment, the treatment comprises an antiviral agent, optionally with an interferon.

Preferably said antiviral agent is an inhibitor of viral replication, such as ribavirin.

The particular purpose of the present invention is to provide a new genetic approach for predicting the response to viral infection treatment. The present invention now discloses the identification of an antiviral treatment response gene locus, the CTGF gene locus (CCN2), which can be used for predicting the response to antiviral treatment of a patient suffering from viral infection, especially HCV. The invention resides, in particular, in a method which comprises detecting in a sample from the subject the presence of an alteration in the CTGF gene locus (CCN2), the presence of said alteration being indicative of the response to the treatment, i.e. being indicative of a level of risk for the patient not to respond to the treatment

The method of the invention allows for prediction of the response to treatment with an antiviral agent such as ribavirin, and an interferon administered to patient suffering of a viral infection, especially hepatitis C.

### DETAILED DESCRIPTION OF THE INVENTION

This invention provides valuable markers to predict response to antiviral treatment, especially in hepatitis C.

Early identification of responders and non-responders subjects to antiviral treatment, would allow for initiation of an individualized (personalized) treatment based on patients' genotype. This would in turn help physicians to make more informed decision, and avoid needless expenditures and unnecessary side effects. The development of these various early prediction techniques bodes well for the future care of patients with viral infection, especially hepatitis C.

The inventors have now identified a gene associated with response to an antiviral treatment. They have shown that response to the antiviral treatment Ribavirin-IFN in French cohorts infected with HCV is dependent on allelic variants lying in the CTGF gene. In particular, one of these variants (rs9402373), which was shown to be associated with antiviral treatment response in HCV infected subjects, exhibits altered nuclear factor binding. Other SNPs in the CTGF gene are also identified as markers of antiviral treatment response.

The Patient

The patient may be any mammal, preferably a human being, whatever its age or sex. The patient may be infected with a virus, including a virus which is selected from the group consisting of virus of the family of Arenaviridae (e.g. Lassa virus), Coronaviridae (e.g. Sever Acute Respiratory Syndrome virus), Flaviviridae (e.g. Hepatitis C or B Virus, Dengue virus, West Nil Virus, Yellow Fever Virus, Tick-Borne Encephalitis virus), Filoviridae (e.g. Ebola, Marburg), Herpesviridae (e.g. Herpes Simplex Virus, Cytomegalovirus, Epstein-Barr Virus, Varicella Zoster Virus), Orthomyxoviridae (e.g. Influenza A and B), Paramyxoviridae (e.g. Respiratory Syncytial Virus, Parainfluenza Virus, PMV, Measles), Poxviridae (e.g. Vaccinia, Variola), Rhabdoviridae (e.g. Vesicular Stomatitis Virus, Viral Hemorrhagic Septicemia Virus, Rabies), Retroviridae (e.g. HIV and other retroviruses), Togaviridae (e.g. Chikungunya, Sindbis, Semliki Forest Virus, Ross River Virus, Eastern Equine Encephalitis Virus). In a particular embodiment, the patient is infected with a Hepatitis C virus, e.g. Hepatitis C virus of genotype 1.

The “sample” may be any biological sample derived from a patient, which contains nucleic acids or polypeptides. Examples of such samples include fluids, tissues, cell samples, organs, biopsies, etc. Most preferred samples are blood, plasma, saliva, urine, seminal fluid, etc. The sample may be collected according to conventional techniques and used directly for diagnosis or stored.

In a particular embodiment, “viral infection” designated all types of human viral infection which may be treated with Ribavirin and/or IFN, for examples hepatitis C, hepatitis B, Respiratory Syncytial Virus (RSV) bronchiolitis, adenovirus disease, influenza and any human viral infection treating with Ribavirin and/or IFN.

Within the context of this invention, “responder” refers to the phenotype of a patient who responds to the treatment with an antiviral agent, especially Ribavirin, and/or an IFN, i.e. the viral load is decreased, at least one of his symptoms is alleviated, or the development of the disease is stopped, or slowed down.

Within the context of this invention, “non-responder” refers to the phenotype of a patient who does not respond to the treatment with an antiviral, especially Ribavirin, and/or an IFN, i.e. the viral load does not substantially decrease, or his symptoms are not alleviated, or the disease progresses.

The Treatment

The term “treatment” or “antiviral treatment” refers to administration of an antiviral agent and/or interferons (IFN).

Preferably the interferon is interferon gamma. However other interferons are encompassed, including interferon alpha 2B, pegylated interferon alpha, consensus interferon, interferon alpha 2A, and lymphoblastoid interferon tau.

In a preferred embodiment, the interferon is PEGylated interferon, such as PEGylated interferon gamma.

The “antiviral agent” may be any compound that interferes with the virus entry into a cell, or its replication, or inhibits the activity of a viral protein.

For instance it may be interfering RNA, anti-sense RNA, Imigimod, ribavirin, an inosine 5′-monophospate dehydrogenase inhibitor, amantadine, or rimantadine. More generally it may be a viral protease inhibitor.

When the virus is HCV virus, the viral agent may be an inhibitor of HCV metalloprotease, HCV serine protease, HCV polymerase, HCV helicase, HCV NS4B protein, HCV entry, HCV assembly, HCV egress, or HCV NS5A protein.

In a preferred aspect, the interferon is interferon gamma, such as PEGylated interferon gamma.

In another preferred aspect, the interferon is interferon alpha, such as PEGylated interferon alpha.

In a specific embodiment, the treatment comprises ribavirin and interferon gamma or alpha, preferably PEGylated interferon gamma or alpha.

The CTGF Gene Locus

Within the context of this invention, “the CTGF gene locus” (Connection Tissue Growth Factor), also called CCN2 gene locus, designates all sequences or products in a cell or organism, including CTGF coding sequences, CTGF non-coding sequences (e.g., introns), CTGF regulatory sequences controlling transcription and/or translation (e.g., promoter, enhancer, terminator, etc.), all corresponding expression products, such as CTGF RNAs (e.g., mRNAs) and CTGF polypeptides (e.g., a pre-protein and a mature protein); as well as surrounding sequences of 20 kb region, preferably 15.3 kb region, upstream the starting codon of the CTGF gene and 20 kb region, preferably 14.1 kb region, downstream the untranslated region (3′UTR). In a particular embodiment most alterations are not necessarily in the promoter sequence.

Alterations

The alteration may be determined at the level of the CTGF DNA, RNA or polypeptide. Optionally, the detection is performed by sequencing all or part of the CTGF gene locus or by selective hybridization or amplification of all or part of the CTGF gene locus. More preferably a CTGF gene locus specific amplification is carried out before the alteration identification step. An alteration in the CTGF gene locus may be any form of mutation(s), deletion(s), rearrangement(s) and/or insertions in the coding and/or non-coding region of the locus, alone or in various combination(s). Mutations more specifically include point mutations. Deletions may encompass any region of two or more residues in a coding or non-coding portion of the gene locus, such as from two residues up to the entire gene or locus. Typical deletions affect smaller regions, such as domains (introns) or repeated sequences or fragments of less than about 50 consecutive base pairs, although larger deletions may occur as well. Insertions may encompass the addition of one or several residues in a coding or non-coding portion of the gene locus. Insertions may typically comprise an addition of between 1 and 50 base pairs in the gene locus. Rearrangement includes inversion of sequences. The CTGF gene locus alteration may result in the creation of stop codons, frameshift mutations, amino acid substitutions, particular RNA splicing or processing, product instability, truncated polypeptide production, etc. The alteration may result in the production of a CTGF polypeptide with altered function, stability, targeting or structure. The alteration may also cause a reduction in protein expression or, alternatively, an increase in said production.

In a preferred embodiment, said alteration is a mutation, an insertion or a deletion of one or more bases. In a particular embodiment of the method according to the present invention, the alteration in the CTGF gene locus is selected from a point mutation, a deletion and an insertion in the CTGF gene or corresponding expression product, more preferably a point mutation and a deletion. The alteration may be determined at the level of the CTGF DNA, RNA or polypeptide.

In a preferred embodiment, said alteration is located within 20 kb, upstream the start codon of the CTGF gene and 20 kb, downstream the 3′UTR of the CTGF gene.

Preferably, the alteration lies in the surrounding sequences of 15.3 kb region, upstream the starting codon of the CTGF gene and 14.1 kb region, downstream the untranslated region (3′UTR).

The method of the invention may preferably comprise determining SNP rs6918698 and/or SNP rs9402373.

TABLE 1A
Antiviral treatment response-associated alterations in the CTGF gene locus

| Nucleotide position in | | | |
|---|---|---|---|
| genomic sequence of | Alteration/SNP | | Sequence |
| chromosome | reference | Polymorphism | reference |
| 132314950 | rs6918698 | C/G | SEQ ID NO: 1 |
| 132319124 | rs9402373 | C/G | SEQ ID NO: 2 |

The presence of a C allele with respect to SNP rs9402373, more particularly of a CC genotype, is deleterious for the patient positive response to the antiviral treatment, i.e. it is indicative of a patient being indicative of a patient being non-responder to said treatment.

The CC or GG genotype with respect to SNP rs6918698 is deleterious for the patient positive response to the antiviral treatment

In another embodiment the method of the invention may comprise determining SNP is selected from the group consisting of SNP rs9388949, SNP rs7748518, SNP rs4897554, and SNP rs928505.

In another embodiment the method of the invention may comprise determining the presence of a deletion of bases as defined in rs3037970.

More generally the method of the invention may comprise determining whether the patient comprises a genotype of non-response as defined in Table 1B.

TABLE 1B
Further antiviral treatment response-associated
alterations in the CTGF gene locus

| SNP in | Genotype of non- | | Sequence |
|---|---|---|---|
| correlation | response | position | reference |
| rs7748518 | GG | 132318911 | SEQ ID NO: 3 |
| rs4897554 | GG, AA | 132304684 | SEQ ID NO: 4 |
| rs9388949 | TT, CC | 132319255 | SEQ ID NO: 5 |
| rs928505 | CC, TT | 132339834 | SEQ ID NO: 6 |
| rs3037970 | -/-, TAAAA/TAAAA | | SEQ ID NO: 7 |

The presence of a G allele with respect to SNP rs7748518, more particularly of a GG genotype, is deleterious for the patient positive response to the antiviral treatment.

The presence of GG or AA genotypes with respect to SNP rs4897554, is deleterious for the patient positive response to the antiviral treatment.

The presence of a TT or CC genotypes at with respect to SNP rs9388949, is deleterious for the patient positive response to the antiviral treatment.

The presence of CC or TT genotypes with respect to SNP rs928505, is deleterious for the patient positive response to the antiviral treatment. The presence of the homozygous genotypes (−/− or TAAAA/TAAAA) at position of the deletion rs 3037970, is deleterious for the patient positive response to the antiviral treatment.

Alterations in the CTGF gene may be detected by determining the presence of an altered CTGF RNA expression. Altered RNA expression includes the presence of an altered RNA sequence, the presence of an altered RNA splicing or processing, the presence of an altered quantity of RNA, etc. These may be detected by various techniques known in the art, including by sequencing all or part of the CTGF RNA or by selective hybridisation or selective amplification of all or part of said RNA, for instance.

In a further variant, the method comprises detecting the presence of an altered CTGF polypeptide expression. Altered CTGF polypeptide expression includes the presence of an altered polypeptide sequence, the presence of an altered quantity of CTGF polypeptide, the presence of an altered tissue distribution, etc. These may be detected by various techniques known in the art, including by sequencing and/or binding to specific ligands (such as antibodies), for instance.

Sequencing

Sequencing can be carried out using techniques well known in the art, using automatic sequencers. The sequencing may be performed on the complete CTGF gene locus or, more preferably, on specific domains thereof, typically those known or suspected to carry deleterious mutations or other alterations.

Amplification

Amplification is based on the formation of specific hybrids between complementary nucleic acid sequences that serve to initiate nucleic acid reproduction. Amplification may be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Preferred techniques use allele-specific PCR or PCR-SSCP. Amplification usually requires the use of specific nucleic acid primers, to initiate the reaction. Nucleic acid primers useful for amplifying sequences from the CTGF gene locus are able to specifically hybridize with a portion of the CTGF gene locus that flank a target region of said locus, said target region being altered in non responder patients. Another particular object of this invention resides in a nucleic acid primer useful for amplifying sequences from the CTGF gene or locus including surrounding regions. Such primers are preferably complementary to, and hybridize specifically to nucleic acid sequences in the CTGF gene locus. Particular primers are able to specifically hybridize with a portion of the CTGF gene locus that flank a target region of said locus, said target region being altered in non responders. Primers that can be used to amplify CTGF target region comprising SNPs as identified in Table 2 may be designed based on their sequence or on the genomic sequence of CTGF.

The invention also relates to a nucleic acid primer, said primer being complementary to and hybridizing specifically to a portion of a CTGF gene locus coding sequence (e.g., gene or RNA) altered in certain non responders subjects. In this regard, particular primers of this invention are specific for altered sequences in a CTGF gene locus or RNA. By using such primers, the detection of an amplification product indicates the presence of an alteration in the CTGF gene locus. In contrast, the absence of amplification product indicates that the specific alteration is not present in the sample. The invention also concerns the use of a nucleic acid primer or a pair of nucleic acid primers as described above in a method of determining antiviral treatment response of infected subjects or in a method of assessing the response of a subject to a treatment of a viral infection.

Selective Hybridization

Hybridization detection methods are based on the formation of specific hybrids between complementary nucleic acid sequences that serve to detect nucleic acid sequence alteration(s). A particular detection technique involves the use of a nucleic acid probe specific for wild-type or altered CTGF gene or RNA, followed by the detection of the presence of a hybrid. The probe may be in suspension or immobilized on a substrate or support (as in nucleic acid array or chips technologies). The probe is typically labeled to facilitate detection of hybrids. In this regard, a particular embodiment of this invention comprises contacting the sample from the subject with a nucleic acid probe specific for an altered CTGF gene locus, and assessing the formation of an hybrid. In a particular preferred embodiment, the method comprises contacting simultaneously the sample with a set of probes that are specific, respectively, for wild type CTGF gene locus and for various altered forms thereof. In this embodiment, it is possible to detect directly the presence of various forms of alterations in the CTGF gene locus in the sample. Also, various samples from various subjects may be treated in parallel.

Within the context of this invention, a probe refers to a polynucleotide sequence which is complementary to and capable of specific hybridization with a (target portion of a) CTGF gene or RNA, and which is suitable for detecting polynucleotide polymorphisms associated with CTGF alleles which predispose to or are associated with reduced antiviral treatment response. Probes are preferably perfectly complementary to the CTGF gene, RNA, or target portion thereof. Probes typically comprise single-stranded nucleic acids of between 8 to 1000 nucleotides in length, for instance of between 10 and 800, more preferably of between 15 and 700, typically of between 20 and 500. It should be understood that longer probes may be used as well. A preferred probe of this invention is a single stranded nucleic acid molecule of between 8 to 500 nucleotides in length, which can specifically hybridize to a region of a CTGF gene locus or RNA that carries an alteration.

The method of the invention employs a nucleic acid probe specific for an altered (e.g., a mutated) CTGF gene or RNA, i.e., a nucleic acid probe that specifically hybridizes to said altered CTGF gene or RNA and essentially does not hybridize to a CTGF gene or RNA lacking said alteration. Specificity indicates that hybridization to the target sequence generates a specific signal which can be distinguished from the signal generated through non-specific hybridization. Perfectly complementary sequences are preferred to design probes according to this invention. It should be understood, however, that certain mismatch may be tolerated, as long as the specific signal may be distinguished from non-specific hybridization.

Particular examples of such probes are nucleic acid sequences complementary to a target portion of the genomic region including the CTGF gene locus or RNA carrying a point mutation as listed in Table 1 above. More particularly, the probes can comprise a sequence selected from the group consisting of SEQ ID NO 1 to 8 or a fragment thereof comprising the SNP or a complementary sequence thereof.

The sequence of the probes can be derived from the sequences of the CTGF gene and RNA as provided in the present application. Nucleotide substitutions may be performed, as well as chemical modifications of the probe. Such chemical modifications may be accomplished to increase the stability of hybrids (e.g., intercalating groups) or to label the probe. Typical examples of labels include, without limitation, radioactivity, fluorescence, luminescence, enzymatic labelling, etc. The invention also concerns the use of a nucleic acid probe as described above in a method of determining antiviral treatment response of HCV infected subjects or in a method of assessing the response of a subject to a treatment of a viral infection.

Specific Ligand Binding

As indicated above, alteration in the CTGF gene locus may also be detected by screening for alteration(s) in CTGF polypeptide sequence or expression levels. In this regard, contacting the sample with a ligand specific for a CTGF polypeptide and determining the formation of a complex is also described. Different types of ligands may be used, such as specific antibodies. In a specific embodiment, the sample is contacted with an antibody specific for a CTGF polypeptide and the formation of an immune complex is determined. Various methods for detecting an immune complex can be used, such as ELISA, radio-immunoassays (RIA) and immuno-enzymatic assays (IEMA). Within the context of this invention, an antibody designates a polyclonal antibody, a monoclonal antibody, as well as fragments or derivatives thereof having substantially the same antigen specificity. Fragments include Fab, Fab′2, CDR regions, etc. Derivatives include single-chain antibodies, humanized antibodies, poly-functional antibodies, etc. An antibody specific for a CTGF polypeptide designates an antibody that selectively binds a CTGF polypeptide, i.e., an antibody raised against a CTGF polypeptide or an epitope-containing fragment thereof. Although non-specific binding towards other antigens may occur, binding to the target CTGF polypeptide occurs with a higher affinity and can be reliably discriminated from non-specific binding.

It is also disclosed a kit to predict treatment response comprising products and reagents for detecting in a sample from a subject the presence of an alteration in the CTGF gene locus or polypeptide, in the CTGF gene or polypeptide expression, and/or in CTGF activity. Said kit comprises any primer, any pair of primers, any nucleic acid probe and/or any ligand, preferably antibody, described in the present invention. Said kit can further comprise reagents and/or protocols for performing a hybridization, amplification or antigen-antibody immune reaction.

Linkage Disequilibirum

Once a first SNP has been identified in a genomic region of interest, more particularly in CTGF gene locus, other additional SNPs in linkage disequilibrium with this first SNP can be identified. Indeed, any SNP in linkage disequilibrium with a first SNP associated with non-responder phenotype will be associated with this trait. Therefore, once the association has been demonstrated between a given SNP and non-responder phenotype, the discovery of additional SNPs associated with this trait can be of great interest in order to increase the density of SNPs in this particular region. Identification of additional SNPs in linkage disequilibrium with a given SNP involves: (a) amplifying a fragment from the genomic region comprising or surrounding a first SNP from a plurality of individuals; (b) identifying of second SNP in the genomic region harboring or surrounding said first SNP; (c) conducting a linkage disequilibrium analysis between said first SNP and second SNP; and (d) selecting said second SNP as being in linkage disequilibrium with said first marker. Sub-combinations comprising steps (b) and (c) are also contemplated. These SNPs in linkage disequilibrium can also be used in the methods according to the present invention, and more particularly in the methods to predict treatment response according to the present invention.

Causal Mutation

Mutations in the CTGF gene locus which are responsible for non-responder phenotype may be identified by comparing the sequences of the CTGF gene locus from patients presenting non-responder phenotype and responder phenotype. Based on the identified association of SNPs of CTGF, the identified locus can be scanned for mutations. In a preferred embodiment, functional regions such as exons and splice sites, promoters and other regulatory regions of the CTGF gene locus are scanned for mutations. Preferably, patients presenting non-responder phenotype carry the mutation shown to be associated with non-responder phenotype and responder phenotype do not carry the mutation or allele associated with reduced antiviral treatment response. The method used to detect such mutations generally comprises the following steps: amplification of a region of the CTGF gene locus comprising a SNP or a group of SNPs associated with non responder phenotype from DNA samples of the CTGF gene locus from patients presenting non responder phenotype and responder phenotype; sequencing of the amplified region; comparison of DNA sequences of the CTGF gene from patients presenting non responder phenotype and responder phenotype; determination of mutations specific to patients presenting non responder phenotype.

Treatment

It is further provided a method for treating a viral infection in a patient in need thereof, which method comprises administering an antiviral agent and/or interferon in a patient that has been tested as responder according to the method described above.

Further aspects and advantages of the present invention will be disclosed in the following experimental section, which should be regarded as illustrative and not limiting the scope of the present application.

### EXAMPLE 1

### Association of two SNPs with Response to Antiviral Treatment

Material and Methods

Subjects

Genotyping was performed on French subjects (n=122) infected with HCV genotype 1 or 4 and treated with antiviral agent Ribavirin and peggylated IFN.

Statistical Analysis

Multivariate logistic regression was used to analyse the relationship between the probability of an individual having reduced antiviral treatment response and genetic variants including the main covariates known to affect antiviral treatment response in subjects infected with HCV. The statistical SPSS software (version 10.0) was used for this analysis

DNA Extraction

Aliquots of 5 to 15 ml of blood were collected on sodium citrate and kept at −20° C. DNA was extracted using the standard salting out method (Sambrook et al., 1989). Some subjects refused bleeding. In this case, buccal cell samples were collected using foam-tipped applicators and applied to indicating FTA1 cards following the protocol described by Whatman (http://www.whatman.co.uk/).

DNA Amplification

All the DNA purified from FTA card were pre amplified before genotyping. Polymerase chain reactions (whole genome amplifications) were conducted in 50 μl reactions containing one punch of biological sample (FTA1-bound buccal cell DNA) or 100 ng of genomic DNA, 1.5 OD of 15-base totally degenerate random primer (Genetix, Paris, France), 200 mM dNTPs, 5 mM MgCl₂, 5 ml of 10× PCR buffer and 0.5 unit of high fidelity Taq DNA polymerase (BIOTAQ DNA Polymerase, Bioline London, England). Samples were amplified in a multiblock thermocycler as follows: a pre-denaturation step of 3 min at 94° C., 50 cycles consisting of 1 min at 94° C., 2 min at 37° C., 1 min of ramp (37-55° C.), and 4 min at 55° C. Final extension step was carried out 5 min at 72° C.

Sequencing

Purified PCR products were sequenced using ABI Prism BigDye Terminator cycle sequencing system (PE Applied Biosystems, Foster City, U.S.A.) on ABI Prism automatic sequencer. Sequencing reactions were performed on both strands Sequencing by GATC biotech (GATC, Marseille France).

Polymorphism Genotyping by PCR with Specific TaqMan Probes

Allelic discrimination was assessed using TaqMan probe assays (Applied Biosystems, Lafayette USA). Each reaction contained 12.5 ng of genomic DNA, TaqMan Universal PCR Master Mix (Applied Biosystems, Lafayette USA), 900 nM of each primer and 200 nM of each fluorescently-labelled hybridisation probe in a total volume of 5 μl. RT-PCR was conducted in an ABI Prism Sequence Detection System 7900 (Applied Biosystems, Lafayette USA) using the following conditions: 50° C. for 2 min, 95° C. for 10 min and 40 cycles of amplification (95° C. denaturation for 15 s, 60° C. annealing/extension for 1 min).

Nuclear Extract Preparation

Nuclear extracts were prepared from human hepatocyte cell line (HEPG2) stimulated for one hour with dexamethasone (1 mM) since hepatocytes produce CTGF in hepatic fibrosis (HF) Kobayashi et al., 2005, Gressner et al., 2007) together with hepatic stellate cells, and endothelial cells and myo-fibroblasts (Gressner et al., 2008). The extracts were prepared with the nuclear and cytoplasmic extraction reagents from Pierce (NE-PER; Pierce, Rockford, Ill., USA).

Electrophoretic Mobility Shift Assay (EMSA)

Complementary single-stranded oligonucleotides were commercially synthetised to span approximately 10 bp on either side of the variant nucleotide, as follows:

(SEQ ID NO: 8)
rs9402373C GCTCTCAAAACTAAGCCCAACTC
(SEQ ID NO: 9)
rs9402373G GAGTTGGGCTTAGTTTTGAGAGC

Complementary strands were annealed by placing reactions (oligonucleotide sens and oligonucleotide antisens) in boiling water for 10 min and allowing to cool to room temperature. Binding reactions were set up with LightShift Chemiluminescent EMSA Kit (Pierce, Rockford, Ill., USA). Aliquots of 20 fmol of complementary DNA were incubated at room temperature for 20 min with 4 mg of nuclear extract in 10 mM Tris, 50 mM KCl, 1 mM DTT, 2.5% glycerol, 5 mM MgCl₂, 50 ng/pl poly d(I-C), 0.05% NP-40, pH 7.5. Then reactions were loaded onto an 8% non-denaturing polyacrylamide gel and run for 150 min at 110V. Free DNA and DNA/protein complexes were transferred to nylon N+membrane by capillary action. Binding was detected according to manufacturer's instructions (Pierce, Rockford, Ill., US.

Results

Results show that the response to treatment is reduced in patients with SNP rs 691898 GG,CC (OR=2.5, Cl=1.2-5.3) and SNP rs 9402373 CC (OR=2.2, Cl=1-5) genotype.

Multivariate analysis shows that the two SNPs affect the treatment response independently from one another.

### EXAMPLE 2

### Association of Other SNPs with Response to Antiviral Treatment

The inventors selected SNPs in the CCN2 gene with an allele frequency greater than 10% and grouped the selected SNPs in 6 correlation groups (r2> or equal to 80%). They tested the association with response to treatment of one representative SNP (eventually two) per correlation group; gender was a significant (p=0.05) covariate in this analysis. The inventors found that SNPs in two correlation groups (III and VI) were associated with response to treatment. In group III, the deletion rs3037970, both homozygous genotypes,(p=0.03 OR=2.3 Cl=0.09-3.54) and rs6918698 GG, CC (p=0.017, OR=2.5, Cl=1.18-5.3) showed an association with the non responder phenotype (no response to Ribavirin +IFN treatment). In group VI, rs9402373 CC was associated (p=0.04, OR=2.27, Cl=1.03-4.98) with the non-responder status.

Then, the inventors tested whether the associations observed with SNPs in the correlation groups III and VI were independent by performing a multivariate analysis including gender as covariate. They found that the associations of the SNPs in these two groups were independent; the best statistical model included rs6918698 (p=0.04) and rs9402373 (p=0.05) and gender (p=0.05). The combined ODD ratio was 4.88 for the individuals who carried the non responder genotypes for both SNPs. See Table 3.

Any SNP correlated (r2>0.6) with rs9402373 and rs6918698 is a marker associated with response to treatment. These other SNPs (rs9388949, rs7748518, rs4897554, rs928505) as well as the deletion rs3037970, may also be considered as markers of response/no response to treatment.

TABLE 2
Association with response to anti-viral treatment in French cohort
French Sample

| | | | | | | Non | | | |
|---|---|---|---|---|---|---|---|---|---|
| | | | | GENOTYPE | responder | responder | | | |
| Analysis | SNP | Position | Bins | (aggravating)² | % | % | OR | 95% Cl | P |
| Univariate | 1257705 | 132304944 | II | | | | | | NS |
| | 12526196 | 132305169 | II | | | | | | NS |
| | 9399005 | 132310657 | | | | | | | NS |
| | 3037970 | 132316891 | III | -/-, TAAAA/TAAAA | 42.4 (28) | 63.6 (35) | 2.3 | 1.09-3.54 | 0.03 |
| | 6918698 | 132314950 | III | GG+CC | 46.3 (31) | 67.3 (37) | 2.5 | 1.18-5.3 | 0.017 |
| | 1931002 | 132320175 | IV | | | | | | NS |
| | 2151532 | 132316423 | IV | | | | | | NS |
| | 9402373 | 132319124 | VI | CC | 56.1 (37) | 74.5 (41) | 2.27 | 1.03-4.98 | 0.04 |
| | 12527379 | | | | | | | | NS |
| Multivariate | 6918698 | | | GG+CC | | | 2.22 | 1-4.8 | 0.04 |
| | 9402373 | | | CC | | | 2.2 | .98-4.9 | 0.05 |

Covariate Sex p =0.05

TABLE 3
SNPs in correlation

| | SNP in | | Genotype of | |
|---|---|---|---|---|
| Group | correlation | r2 | non-response | position |
| III | rs7748518 | 1.0 | GG | 132318911 |
| | rs9402373 | 0.7 | CC | 132319124 |
| VI | rs6918698 | 1.0 | GG, CC | 132314950 |
| | rs4897554 | 0.677 | GG, AA | 132304684 |
| | rs9388949 | 0.86 | TT, CC | 132319255 |
| | rs928505 | 0.697 | CC, TT | 132339834 |
| | rs3037970 | | -/-, TAAAA/TAAAA | |

### REFERENCES

Czaja A, Freese D K. Diagnosis and Treatment of Autoimmune hepatitis. Hepatology 2003; 473-496.

Poupon R E, Lindor K D, Cauch-Dudek K, et al. Combined analysis of randomized controlled trials of ursodeoxycholic acid in primary biliary cirrhosis. Gastroenterology 1997; 113:884.

Bruck R, Shirin H, Hershkoviz R, et al. Analysis of Arg-Gly-Asp mimetics and soluble receptor of tumour necrosis factor as therapeutic modalities for concanavalin A induced hepatitis in mice. Gut 1997; 40:133.

Schuppan D, Krebs A, Bauer M and Hahn E G, Hepatitis C and liver fibrosis, Cell Death and Differenciation (2003) 10, S59-S67

Nathan D G and Orkin S H, Musings and genome medicine: Hepatitis C, Genome medicine 2010, 2:4

Fried M W, Side effects of therapy of Hepatitis C and their management, HEPATOLOGY, Vol. 36, No. 5, Suppl. 1, 2002

1. An in vitro method for determining the likelihood of a patient affected with a viral infection to respond to a treatment with an antiviral agent and/or an interferon, which method comprises determining alteration in CTGF gene locus or in CTGF expression or CTGF protein activity in a biological sample of the patient.

2. The method of claim 1, wherein said alteration is a mutation, an insertion or a deletion of one or more bases.

3. The method of claim 2, wherein said alteration is one or several single nucleotide polymorphism(s) (SNPs).

4. The method of claim 3, wherein said SNP is rs9402373 (SEQ ID NO:1) and the presence of a CC genotype of rs9402373 is indicative of a patient being non-responder to said treatment.

5. The method of claim 3, wherein said SNP is rs6918698 (SEQ ID NO: 2), and the presence of CC or GG genotype of rs6918698 is indicative of a patient being non-responder to said treatment.

6. The method of claim 3, wherein said SNP is selected from the group consisting of rs9388949, rs7748518, rs4897554, and rs928505.

7. The method of claim 2, wherein said alteration is a deletion of bases as defined in rs3037970.

8. The method of claim 1, wherein the treatment comprises an antiviral agent, optionally with an interferon.

9. The method according claim 1, wherein said antiviral agent is an inhibitor of viral replication.

10. The method of claim 9, wherein said antiviral agent is ribavirin.

11. The method of claim 1, wherein the antiviral agent is a viral protease inhibitor.

12. The method according to claim 1, wherein the interferon is interferon gamma.

13. The method according to claim 1, wherein the interferon is interferon alpha.

14. The method of claim 1, wherein the interferon is PEGylated interferon gamma or alpha.

15. The method of claim 1, wherein the treatment comprises ribavirin and interferon gamma or alpha, preferably PEGylated interferon gamma or alpha.

16. The method of claim 1, wherein the patient is affected with a virus which is selected from the group consisting of virus of the family of Arenaviridae (e.g. Lassa virus), Coronaviridae (e.g. Sever Acute Respiratory Syndrome virus), Flaviviridae (e.g. Hepatitis C Virus, Dengue virus, West Nil Virus, Yellow Fever Virus, Tick-Borne Encephalitis virus), Filoviridae (e.g. Ebola, Marburg), Herpesviridae (e.g. Herpes Simplex Virus, Cytomegalovirus, Epstein-Barr Virus, Varicella Zoster Virus), Orthomyxoviridae (e.g. Influenza A and B), Paramyxoviridae (e.g. Respiratory Syncytial Virus, Paralnfluenza Virus, PMV, Measles), Poxviridae (e.g. Vaccinia, Variola), Rhabdoviridae (e.g. Vesicular Stomatitis Virus, Viral Hemorrhagic Septicemia Virus, Rabies), Retroviridae (e.g. HIV and other retroviruses), Togaviridae (e.g. Chikungunya, Sindbis, Semliki Forest Virus, Ross River Virus, Eastern Equine Encephalitis Virus).

17. The method of claim 1, wherein the virus is hepatitis C virus.

18. The method of claim 16, wherein the virus is hepatitis B virus.

19. The method of claim 1, wherein the presence of an alteration in the CTGF gene locus is detected by sequencing, selective hybridization and/or selective amplification.


SEQUENCE LISTING (10 sequences)

SEQ ID NO: 1 - DNA, 601 bp, Homo sapiens
  misc_feature (301)..(301): S=C/G
ccccccaacccttagcaatgatttgcgttttagaggctataggctcttgaaactctccaaagagtagaaaaggagtgagttgcttttaaccatttgaaatcctcaagatgcctacctgtaaaaccctgaattctatatttgagatttgatcatttgtcacttgaggtaacggttttgggacaagaaagagaacaaagacgcgtgtgactcaggatgcagtctcctggggcagatttccaaaactcttgcttagttttctcttaatatatgtaggagttttatataggcaaggacaagggasgagtggccatcaatgtttccagaaaaagggatccattgttctatcagagcaaatgattctgtgttgggtaggtaggggctctgggtgtcagggtgggaacactgggatgcaaagggggttcacctaggagcatttaaacacagcttcactagggtctttgagaaatgagtgcattttcttatattaaaacctataatcttcaaatgtggattggatttggtttgtcttgttaaaataagaagtctgattccattacaagagcagtgtaccctgttgtatctggatataaactaagattga

SEQ ID NO: 2 - DNA, 817 bp, Homo sapiens
  misc_feature (317)..(317): S=C/G
cccatcattcatccctggaaatgtgctcccacctctagccaagaggcctgaggtcccttctcacaacttcaaactccctggaataccattaggtggagcctaggggactggctaggaggtgaaggtgtgagctccatacctgccatttgcttgccagtgacctcagggaagacacctactctctctgagcctgaaatttcccacctgttcacagttaatgtgcagctcaaggagacaatatttatgaaagcactttcataaaggtattagagttttaaaattgcccttctcttccattcttcagtggctctcaaaastaagcccaactcagattgactgtaaaagcctgtcacagtttgattttataacagtgtccagctctgcatgggttttagagtgttaaaactaggtgagagctcagagaaaatcaaatcctacctcttatttatttatttattttattagattagatattcaggattgatagagacatttaacaaatgaaaactttttttctttttgaaatagggtctggctctgtctcccagggtggaatgtagtgccatgatcttggctcactgcaacctctgcctcctgggctcaagtgatcttcctgcctcagcctcccaagtagctgggatagctgggactacaggtgtgcaccaccatatccagctaattaaaagaatttttttttttttttttgtagagatggggttttgccatgttgcccaggctggtctcgaactcctgagctcaagagatcagcccatctcagcctcccaaagtgctgggattacaggcgtaagcccctgcg

SEQ ID NO: 3 - DNA, 501 bp, Homo sapiens
  allele (251)..(251): r=a/g
agagagaattgggactagctatcttatctcaaataatagaagtaagtgaaaggataaatattttgaaaggactagtgtgaaatatacatgaatgtattcctagggattctgctgcggggtgggaaaggggtgcagattaagccagaccccatcattcatccctggaaatgtgctcccacctctagccaagaggcctgaggtcccttctcacaacttcaaactccctggaataccattaggtggagcctagrggactggctaggaggtgaaggtgtgagctccatacctgccatttgcttgccagtgacctcagggaagacacctactctctctgagcctgaaatttcccacctgttcacagttaatgtgcagctcaaggagacaatatttatgaaagcactttcataaaggtattagagttttaaaattgcccttctcttccattcttcagtggctctcaaaactaagcccaactcagattgactgtaaaagcctgtcaca

SEQ ID NO: 4 - DNA, 1982 bp, Homo sapiens
  allele (1730)..(1730): r=a/g
ggaactcatcttaaatatttaaaaataagtttttttttctaattgcaaggaataagtactctttgtaaatgatatggaaagtatggaatataataaagatgaaaaagataactcctagataatcacttttaatgttttggcaaaattctttttaaaaagtgctaaagaaaatgtggcacatatacatcatggaatactatgcatccataaaaaagaatgagttcatgtcctttggagggacatagatgaagctgaaagccatcattcttagcaaactaacccaggaacagaaaaccaaacaccgcatattctcactcataagtgggagttgaacaatgagaacacatggacacagggaggggaacatcacacatgggggcctgttggggggtgggggccaaggaaagggaaaacattaggacaaatacctaatgtatgcagagttaaaatctagatgacgggttgataggtgcagcaaaccaccatggcacgtgtatacctatgtaacaaatctgcacattctgcacatgtatcccagaacttaaagtaaaaaaaaaaaaagtgcttgaatatatttaatttttaaactttttaattgaaatataatagagatttaacaatatgtgccaagttctctcatgcccccacctccaaagtaaccactaccctgcctttatcactatttagttttgcttcctcttcaatttcatatagatgacattatgcagtgtgtatgcttttgtgcctgacttctttcactcaccataatagtgcttttcttttttatatataagagatttattctgagccaaatatgagtgaccatggcccatgacacagccctcaagaggtcctgagaacttgtgcccaaggtggtctgggggcagcttggttttatacattttagagcagcatgagacatcaatcacatccgtttaagaaatacattggtttggtccagatgtggagcgggtggtgggggaaggggaggcttccaggttataggtgaatttaaacattttctggttgacaattggttgagtttgtctcaaaacgtgggatagaggctgggcgcagtggctcacgcctataatcccagcactttgggaggccgaggcgggcggatcacctgagctcagaagttcaagaccagcctgggcaacatggtgaaaccccgtctctattaaaaatacaaaaaatcagcctagtgtggtagtgcatgcctgtaatcccagctactcgggaggctgaggcaggagaatcacttgaacccgggaggcagaggttgcagtgagtcgagatcacgccactgcactccggcctgggcgacagagagagactttgtatcaaaaaaaaaaaaaaaaaaaaaaggcctgggatagatagaaagggaatgttcaggttaagataaacattgtggaaacccaagttcttctgaggtcttatagtggctgcctgtagaggcaagaagtgacaaatgtttcctattcagatctcagttcatctctttacgattgggagagtctggaagaaaaagatctatctatattaatagagattctttagaggtgcaaattttccctcacaaagaacaactttgcggggccatttcaaaatatggcaaagaaacatgttttggggaaatgcactttttaattgatgtatattgttccattatataaatattctatactctatcaatttttctgtttacagaattcagatttttrctttcagtttttggatggatgaaactgctttgagaattttgtatgtcttttgctcacttctcttgaatatataccaaagactaaaagttattgtagttatctaaattatacatgttaacttgctgacaaaaattgtgttagaatttgttgacctaatgtaaaagttttatattcttgcgatgcaaattctaattaatcaatggtgctcctcatttcttactaactcttctttgactctcttttaaacatgta

SEQ ID NO: 5 - DNA, 817 bp, Homo sapiens
  allele (317)..(317): y = c/t
cctctagccaagaggcctgaggtcccttctcacaacttcaaactccctggaataccattaggtggagcctaggggactggctaggaggtgaaggtgtgagctccatacctgccatttgcttgccagtgacctcagggaagacacctactctctctgagcctgaaatttcccacctgttcacagttaatgtgcagctcaaggagacaatatttatgaaagcactttcataaaggtattagagttttaaaattgcccttctcttccattcttcagtggctctcaaaactaagcccaactcagattgactgtaaaagccygtcacagtttgattttataacagtgtccagctctgcatgggttttagagtgttaaaactaggtgagagctcagagaaaatcaaatcctacctcttatttatttatttattttattagattagatattcaggattgatagagacatttaacaaatgaaaactttttttctttttgaaatagggtctggctctgtctcccagggtggaatgtagtgccatgatcttggctcactgcaacctctgcctcctgggctcaagtgatcttcctgcctcagcctcccaagtagctgggatagctgggactacaggtgtgcaccaccatatccagctaattaaaagaatttttttttttttttttgtagagatggggttttgccatgttgcccaggctggtctcgaactcctgagctcaagagatcagcccatctcagcctcccaaagtgctgggattacaggcgtaagcccctgcgccgggccacagatgaaacttttaaatttgtc

SEQ ID NO: 6 - DNA, 1659 bp, Homo sapiens
  allele (334)..(334): y=c/t
gaattatctgaaggctcccaaattgtctgcagtatcattacttgctgactgtttttcaccaactacctatctagtttccacagacaagggctcattgtaaaatgtcagaattatcctgctagaaatgcaaaagactcagaaaacttcatacgcatggactctaaatgtttcatgggttttgtttttgtttttgtttacattaagctacagatggggtggagagatgccctgtgatctgtcaggaactgcctgcaaaaactacttgccagctaataattagcattttttcatgggtgctggaaaaagaagtgacattcctgggagaaagaccatygactttattactcttagcaacagcagtagctagcatttcagcagtttcttgcaccagttcgactacctgcacacatagtaagcattataggagagagactccaagcttacagaacctgaattttttatggtgaatagcaagcatgcctgccctttgctctagagagaggcactatctccatcttccaaggctgttcactgtacaaacatttttgaaatgacagaatgaaataatgtcagttagttcctctgctcataagacatgtagaaatgtgagaagcccgtggagaattgtctaccagcatgaccttgacactgagcactcacagcaaacatgtttctattgtgcttggcatcatgctgctagatggtttaaatgactccggatcgccaaggtccttatctggacaaaccactattgcacagtccaaaaatatttttccgagtttctcaagccatctataggtattcatttcctcttttttaaggtcagtaaaatttctgaaacttttgtcttttaccattttccagaataatttggggattcccattacaaaatacttctgcaatgtagaacatctctatttcactagcattaagatagtattccaaaatatggtccataactatcacgggtttgaggtagacataggcttagtgtagaccatgaaagcatttcaataatgatgcttcctgcatctccctcaagtaaacctgttaactcaagttactatttactcaagtaaacctgttaacactgacatgttaacatggtatgaatcctagctttaatgcttactagtttttacttggatgattaacttcttcctctaccttagtctcctcattgtgaaatgggataacaatattcactcttcctaccttacacaatttttgagaaagtcaaatgacataacatttatgaaagcattttataatcaagaaaagatcttaaaggtgctttaatactctaataagtatctcctgcagcttgcttgtgcctcagggatgccaaacctaatcttttcatgccctgagaagctgggttattatagtgtatcaggcatagttgtagggtaatcagttgtttgcaattagattcatgtgttattgatgtccaggggacatatatttaccatcaaggatttagggggaggttgtaaggtaaaatgagtagagagttggagggaatataatgttatttctaggtcagaggccatttctggatatgtaaaatagccaatgttttatgataccacatattggcttttggtgaagttttagctgagattcagtgaataggaaagagataaagcatccttttgt

SEQ ID NO: 7 - DNA, 989 bp, Homo sapiens
gtactgggttatagttagatggcctaggttgtattcttggaactgcctttcgcagatctcagggaagtcacacgtaggagccatattcccatttctgtttaaaaaatgaaagtaatcataatttttttaatttaatagggttcagtgagggctgagtagatgaaaattccagggaattggtgctttatttttttcagattttcttgattttttttcaatctctgtgtataataagtatattaccattctgctctgttgttaaaaatgtcaaaagaaagaatttggttttactataaaattctcatagtccctcgaagtctcaaaaaaatctatagtcagttccaaatagatgattttaatgttgcttaagaggaaataagcatattgaaatatatagaacatatgaaatctcaggaaagaaaaagtttctgtgcattatcaggttagattgaggctttatgatcaaaactctttttgaaggagttaaaataaaagactattattaagagagtgacacagagacagacagacacatacatgcacacacacccagagagagagagaatgagagaagacacccaagacattcacagactcagaagtatgtttttattgtgatatggaccatcaaatgccaggaaactcaaagggaaaggctgatactccatccataattcatgccatgggatctgaattcttgcactattatcaatgactcagcaaatgtttgtgtgtgcctgggcggtaaagtgggaaatagtggcttcttataacttcatccaactttgacttaccccagcccctttgattgacagcaggtcagagttgggagaaaaagatctttgaatttgtgtgcaaaatggttaagatgcactaattttgacttgtaagcaagtatttatctagcaaatgctttttagagagcattttttcctcgtggtaaaatgaggaattaactaagctggccactgagtcctgtttaccaagct

SEQ ID NO: 8 - DNA, 23 bp, Artificial Sequence
  primer
gctctcaaaactaagcccaactc

SEQ ID NO: 9 - DNA, 23 bp, Artificial Sequence
  primer
gagttgggcttagttttgagagc

SEQ ID NO: 10 - DNA, 984 bp, Homo sapiens
gtactgggttatagttagatggcctaggttgtattcttggaactgcctttcgcagatctcagggaagtcacacgtaggagccatattcccatttctgtttaaaaaatgaaagtaatcataatttttttaatttaatagggttcagtgagggctgagtagatgaaaattccagggaattggtgctttatttttttcagattttcttgattttttttcaatctctgtgtataataagtatattaccattctgctctgttgttaaaaatgtcaaaagaaagaatttggttttactataaaattctcatagtccctcgaagtctcaaaaaaatctatagtcagttccaaatagatgattttaatgttgcttaagaggaaataagcatattgaaatatatagaacatatgaaatctcaggaaagaaaaagtttctgtgcattatcaggttagattgaggctttatgatcaaaactctttttgaaggagttaaaagactattattaagagagtgacacagagacagacagacacatacatgcacacacacccagagagagagagaatgagagaagacacccaagacattcacagactcagaagtatgtttttattgtgatatggaccatcaaatgccaggaaactcaaagggaaaggctgatactccatccataattcatgccatgggatctgaattcttgcactattatcaatgactcagcaaatgtttgtgtgtgcctgggcggtaaagtgggaaatagtggcttcttataacttcatccaactttgacttaccccagcccctttgattgacagcaggtcagagttgggagaaaaagatctttgaatttgtgtgcaaaatggttaagatgcactaattttgacttgtaagcaagtatttatctagcaaatgctttttagagagcattttttcctcgtggtaaaatgaggaattaactaagctggccactgagtcctgtttaccaagct